(19) United States
(12) Patent Application Publication
(10) Pub. No.: US 2025/0144046 A1
(43) Pub. Date: May 8, 2025

(54) PHARMACEUTICAL COMPOSITIONS AND METHODS OF ADMINISTRATION

(71) Applicant: METASTX, LLC, Basking Ridge, NJ (US)

(72) Inventors: David CRICH, Athens, GA (US); Somanath SHENOY, Martinez, GA (US); Brian CUMMINGS, Royal Oak, MI (US); Harvey D. HOMAN, Basking Ridge, NJ (US); Daniel A. GESELOWITZ, Bethesda, MD (US)

(21) Appl. No.: 18/833,526

(22) PCT Filed: Jan. 27, 2023
(86) PCT No.: PCT/US2023/061462

§ 371 Date: Jul. 26, 2024

Related U.S. Application Data
(60) Provisional application No. 63/304,058, filed on Jan. 28, 2022.

(51) Int. Cl. A61K 31/10; A61K 31/05; A61P 35/00; C07C 39/14; C07C 323/16; C07C 323/21

(52) U.S. Cl. CPC A61K 31/10; A61K 31/05; A61P 35/00; C07C 39/14; C07C 323/16; C07C 323/21

(57) ABSTRACT

The present disclosure describes novel compounds and compositions and methods of making and using thereof related that are capable of inhibiting PAK1 and associated diseases or disorders, including tumors and cancerous cells. The inventive compounds are more efficacious and stable than IPA-3 without needing liposomal formulations as vehicles for the compounds.

### CLAIM OF PRIORITY

This application claims the benefit of priority to U.S. Provisional Appl. No. 63/304,058, filed Jan. 28, 2022, the contents of which are incorporated by reference herein in their entirety.

### FIELD OF THE TECHNOLOGY

The present disclosure relates to pharmaceutical compounds and compositions and to methods of administration of these compositions for the treatment of diseases or disorders.

### BACKGROUND

Lack of target specificity is a major limitation of many widely used kinase inhibitors. This results in part from the high degree of evolutionary conservation in the ATP-binding pocket targeted by these compounds across the human kinome. Nonconserved regulatory elements found in some kinases may offer unique targets for more selective kinase inhibition.

Most drugs modify the actions of enzymes, receptors, transporters and other molecules by directly binding to their active (orthosteric) sites. However, orthosteric site configuration is similar in several proteins performing related functions and this leads to a lower specificity of a drug for the desired protein. Consequently, such drugs may have adverse side effects.

Activated p21-activated kinase 1 (PAK1) promotes changes in the actin of cells and allows the cytoskeleton to change shape. This allows for a process called epithelial-to-mesenchymal transition (EMT). EMT is one of the key events that enables the cancer cells to migrate, invade and metastasize to distant tissues.

Given the unpredictability in the art of structurally similar anti-cancerous drugs, there is a need for novel compounds that overcome these deficiencies and are efficacious while also being stable without the need for liposomal formulations. These novel compounds would be able to inhibit PAK1/EMT and metastasis and be capable of binding to sites away (allosteric) from the orthosteric sites while having greater specificity than previously known drugs.

### SUMMARY

The present technology overcomes these deficiencies by developing unique compounds that demonstrate PAK1 inhibition and anti-cancer efficacy as well as formulations and methods of making and using said compounds and formulations.

While not being bound by theory, it is believed that Group-1 PAKs (PAK1, PAK2, and PAK3 with a similar mechanism of activation and substrate specificity, and often regulated by tissue-specific expression) are activated downstream of two smaller proteins called GTPases, namely Rac and CDC42. These proteins are necessary for cells to form two processes called lamellipodia and filopodia. Lamellipodia are the membrane protrusions from a cell. Filopodia are spike-like proteins that make new contacts with the substrates. These structures are critical to a cell's ability to change shape and move. Critical to both of these is a protein called actin, which forms the cytoskeleton, or the bones of the cells. Group-I PAK activity is inhibited by IPA-3, a selective, non-ATP competitive PAK1 inhibitor with IC50 of 2.5 μM in a cell-free assay. IPA-3 does not inhibit group II PAKs (PAK4, PAK5, and PAK6), which are different from Group-I PAKs in their mechanism of activation and cellular function.

2,2″-dihydroxy-1, 1′-dinaphthyldisulfide (IPA-3) may be used as a therapeutic agent against cancerous cells by acting as an allosteric PAK1 inhibitor. IPA-3 may trap a transient intermediate step in PAK activation, since pre-activated PAK1 is insensitive to IPA-3. In addition, IPA-3-bound PAK1 exhibits some but not all features of the active conformation and is catalytically inactive. Kinase specificity profiling studies reveal an exceptional degree of kinase selectivity by IPA-3, consistent with its targeting the unique PAK1 regulatory domain. Furthermore, cell-based experiments using IPA-3 provide evidence that PAK promotes mitogen-activated protein kinase activation and illustrate selective inhibition of PAK in live cells. Thus, kinase autoregulatory mechanisms provide an alternative target for kinase inhibition by small molecules.

PAK1 may be essential for the invasion and metastasis of prostate, breast, and several other cancer cell types by promoting the “epithelial-to-mesenchymal-transition” (EMT), a process necessary for the early cancer cells to metastasize. It is believed that suppressing the ability of the cancer cells to migrate and invade adjacent tissues could prevent and/or treat metastatic cancers. IPA-3 may inhibit prostate cancer cell (PCa) EMT and suppress metastasis to other organs. IPA-3 may also stop the growth of metastatic prostate cancer (mPCa) cells in the bone and thus prevent PCa-induced bone remodeling and loss of tissue. Suppression of PAK1 by IPA-3 may prevent PCa cell adhesion and homing in the distant organs, and metastasis.

IPA-3 is unique because of its allosteric nature in suppressing Group-I PAK activity. Furthermore, it does not target the ATP-binding domain of any kinase. Because of these properties, IPA-3 is highly selective in suppressing PAK1 activity in various cell types with minimal effects on other cellular functions.

However, IPA-3 lacks stability in vivo due to the disulfide bridge between the pendant naphthalene groups. Therefore. IPA-3 may be incorporated into formulations that facilitate the delivery and stability of IPA-3 compounds.

One example is lipid-based formulations such as in U.S. Pat. No. 11,517,539, which is incorporated by reference in its entirety.

Surprisingly, the compounds of the present technology advantageously possess efficacy even greater than IPA-3 while also being highly stable without the need of incorporating these compounds into such formulations and without the need for a disulfide bridge, which was previously expected to be necessary for effectiveness.

In one example, the present technology is directed to a compound having the structure:

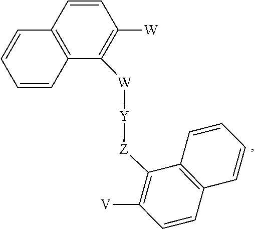

or a pharmaceutically acceptable salt thereof,
wherein W and V are each independently H or OH;
wherein X and Y are each independently an attachment site, methine, methylene, N, N-alkyl, N-acyl, N-sulfonyl, O, S, CO, or SO2, with the provisos that X and Y cannot both be an attachment site, O, S, or SO2 and that if X or Y is methine, X and Y are both methines to form a double bond: wherein the alkyl group of the N-alkyl and N-acyl groups is a C1-C6 alkyl group that is linear or branched, unsubstituted or substituted, with one or more substitutions selected from the group consisting of hydroxyl groups, halogens, cyano groups, amines, and mixtures thereof; and wherein Z is CHR where R is H or C1-C6 alkyl, wherein the C1-C6 alkyl group of Z is linear or branched, unsubstituted or substituted, with one or more substitutions selected from the group consisting of hydroxyl groups, halogens, cyano groups, amines, and mixtures thereof.

In some examples, X is an attachment site (i.e., a bridge with two groups: Y and Z).

In some examples, X is not an attachment site (i.e., a bridge with three groups: X, Y, and Z).

In some examples, the compound is Compound 1 or a pharmaceutically acceptable salt thereof.

In some examples, the compound is Compound 3 or a pharmaceutically acceptable salt thereof.

In some examples, the compound is Compound 4 or a pharmaceutically acceptable salt thereof.

In another example, the present technology incorporates the inventive compounds into a composition. The composition may include the inventive compound(s) and one or more pharmaceutically acceptable excipients.

The compounds of the composition may include an effective amount of the compound(s) of the present technology wherein the effective amount is the minimum amount of the compound(s) in the composition effective to inhibit PAK1 in cells.

In some examples, the composition contains a liposomal formulation. In other examples, the composition does not contain a liposomal formulation.

In another example, the present technology is directed to a method of treating a disease or disorder associated with PAK1.

In some examples, the method treats cancerous cells.

### BRIEF DESCRIPTION OF THE DRAWINGS

The technology will be more fully understood from the following detailed description taken in conjunction with the accompanying drawings, in which:

FIG. 1A shows a bar graph (above) and IC50 curve (below) showing anti-proliferative effects of IPA3 on PC3 cells in vitro compared to vehicle (DMSO)-treated control arm (n=3).

FIG. 1B shows a bar graph (above) and IC50 curve (below) showing anti-proliferative effects of Compound 1 on PC3 cells in vitro compared to vehicle (DMSO)-treated control arm (n=3).

FIG. 1C shows a bar graph (above) and IC50 curve (below) showing anti-proliferative effects of Compound 3 on PC3 cells in vitro compared to vehicle (DMSO)-treated control arm (n=3).

FIG. 1D shows a bar graph (above) and IC50 curve (below) showing anti-proliferative effects of Compound 4 on PC3 cells in vitro compared to vehicle (DMSO)-treated control arm (n=3).

FIG. 2A shows the representative images from a scratch assay (0 and 36 hours) in Vehicle (DMSO), IPA3, Compound 1, Compound 3, and Compound 4 treated PC3 cell monolayers showing the migration of PCa cells after 36 hours.

FIG. 2B shows a bar graph demonstrating the significant inhibitory effect of Compound 3 and Compound 4 on PC3 cell migration compared to DMSO-treated control, IPA3, and Compound 1-treated arms (n=4).

FIG. 3A shows bar graphs demonstrating the apoptosis and necrosis-inducing effects of IPA3, Compound 1, Compound 3, and Compound 4 on PC3 cells in vitro compared to a vehicle (DMSO)-treated control arm (n=3).

FIG. 3B shows corresponding flow cytometry plots demonstrating the cell death-inducing effects of IPA-3 and MTX compounds with a superior effect of Compound 3 and Compound 4 on PC3 cells.

FIG. 4 shows an exemplary embodiment of a synthesis protocol that is used for synthesizing Compound 1.

FIG. 5 shows an alternative exemplary embodiment of a synthesis protocol that is used for synthesizing Compound 1.

FIG. 6 shows an exemplary synthesis embodiment of a synthesis protocol that is used for synthesizing Compound 3.

FIG. 7 shows an exemplary synthesis embodiment of a synthesis protocol that is used for synthesizing Compound 4.

### DETAILED DESCRIPTION

The present technology relates to novel compounds as well as compositions and methods of administration of the same for the treatment of various diseases or disorders.

In an embodiment, the novel compounds are effective against cancerous cells. In a preferred embodiment, the novel compounds are effective against human metastatic prostate cancer (PC3) cells.

The present technology encompasses chemical compounds, compositions containing these compounds, and methods of administration of these compounds to treat disease.

IPA-3 is not very stable in plasma due to the presence of a disulfide bond, which is critical for the inhibition of PAK1, and quickly becomes inactive, thus limiting its therapeutic benefits. Liposomal formulations offer several advantages. They increase the level of drug that gets into the body by helping them avoid degradation. In essence, they increase the stability of the drug. They also decrease the non-target toxicity of the drug as lower levels of the drug can be used. With regard to anti-cancer drugs, liposomes can increase the specificity of the drug to the tumor site. This is due to a phenomenon called the enhanced permeability and retention (EPR) effect. Basically, the increased amount of blood vessels going to the tumor, combined with the leakiness (on increased permeability), allows for liposomes to penetrate the tumor. In contrast, blood vessels to normal tissues are typically not as plentiful and do not have as leaky walls. This means that drugs encapsulated in liposomes selectively aggregate in tumor sites, as opposed to non-cancer tissue.

The nano-liposomal formulation developed by the founders of MetasTx, LLC (“MTX-101”) has demonstrated superior stability and efficacy to inhibit metastatic prostate cancer cells in vitro and in vivo, compared to IPA-3.

Without being bound by theory, the potential advantages of the compounds of the technology include, but are not limited to, the following:

The compounds of the technology may affect one or more mechanisms by which cancerous cells evade the cancer killing-effect of small molecules.

Compared to the currently used taxanes such as Docetaxel (Taxotere) and Cabazitaxel (Jevtana), which both inhibit microtubules and cell proliferation, preclinical studies on the compound of the technology suggest much lower toxicity and side-effects.

### Compounds

The present technology encompasses, but is not limited to, non-IPA-3 compounds as described herein. In some embodiments, IPA-3 is combined with the compounds described herein.

The following are examples of individual structures. The following compounds are for example purposes only and are included in the more expansive generic formulae as described herein. Heterocycles and substituted aromatics of the following compounds are also envisaged.

In an embodiment, the present technology is directed to a compound having the structure:

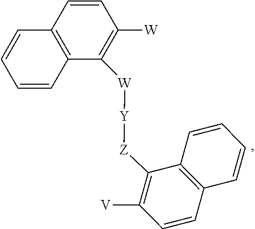

or a pharmaceutically acceptable salt thereof,
wherein W and V are each independently H or OH;
wherein X and Y are each independently an attachment site, methine, methylene, N, N-alkyl, N-acyl, N-sulfonyl, O, S, CO, or SO2, with the provisos that X and Y cannot both be an attachment site, O, S, or SO2 and that if X or Y is methine, X and Y are both methines to form a double bond; wherein the alkyl group of the N-alkyl and N-acyl groups is a C1-C6 alkyl group that is linear or branched, unsubstituted or substituted, with one or more substitutions selected from the group consisting of hydroxyl groups, halogens, cyano groups, amines, and mixtures thereof; and wherein Z is CHR where R is H or C1-C6 alkyl, wherein the C1-C6 alkyl group of Z is linear or branched, unsubstituted or substituted, with one or more substitutions selected from the group consisting of hydroxyl groups, halogens, cyano groups, amines, and mixtures thereof.

In some embodiments, X is an attachment site (i.e., a bridge with two groups: Y and Z).

In some embodiments, X is not an attachment site (i.e., a bridge with three groups: X, Y, and Z).

In some embodiments, X, Y, or Z does not contain a sulfur.

In an embodiment, the compound(s) of the present technology may be in the form of Compound 1:

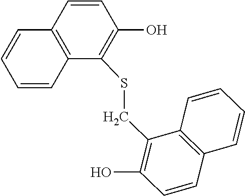 [SMILES: Oc1ccc2ccccc2c1CSc1c(O)ccc2ccccc12]

In an embodiment, the compound(s) of the present technology may be in the form of Compound 2:

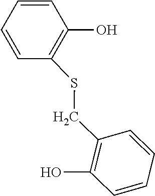 [SMILES: Oc1ccccc1CSc1ccccc1O]

In an embodiment, the compound(s) of the present technology may be in the form of Compound 3:

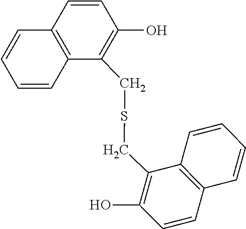 [SMILES: Oc1ccc2ccccc2c1CSCc1c(O)ccc2ccccc12]

In an embodiment, the compound(s) of the present technology may be in the form of Compound 4:

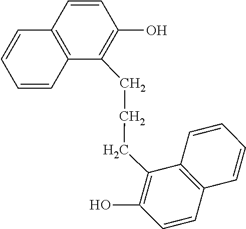 [SMILES: Oc1ccc2ccccc2c1CCCc1c(O)ccc2ccccc12]

In an embodiment, the compound(s) of the present technology may be in the form of Compounds 5 (E-) and 6 (Z—):

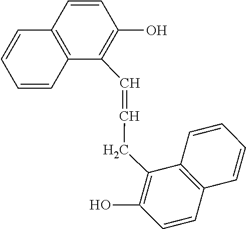 [SMILES: Oc1ccc2ccccc2c1C=CCc1c(O)ccc2ccccc12]

In an embodiment, the compound(s) of the present technology may be in the form of Compound 7:

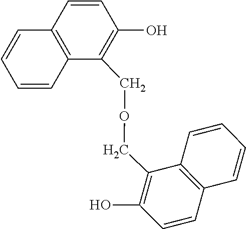 [SMILES: Oc1ccc2ccccc2c1COCc1c(O)ccc2ccccc12]

In an embodiment, the compound(s) of the present technology may be in the form of Compound 8:

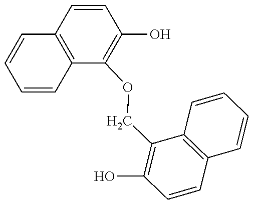 [SMILES: Oc1ccc2ccccc2c1COc1c(O)ccc2ccccc12]

In an embodiment, the compound(s) of the present technology may be in the form of Compounds 9 (X═O), 10 (X═S), 11 (X═SO), 12 (X═SO₂):

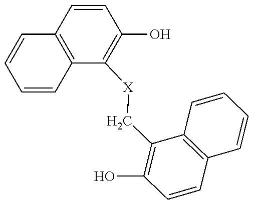

### X═O, S, SO, SO₂

In an embodiment, the compound(s) of the present technology may be in the form of Compounds 13 (X═O), 14 (X═S), 15 (X═SO), 16 (X═SO₂):

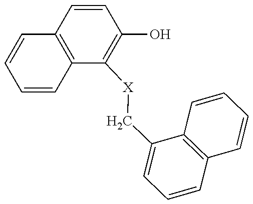

### X═O, S, SO, SO₂

In an embodiment, the compound(s) of the present technology may be in the form of “A” Compounds:

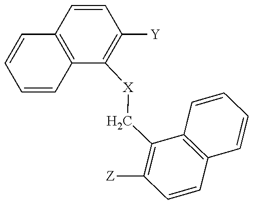

X═O, S, SO, SO₂, Y═Z═OH
X═O, S, SO, SO₂, Y═H, Z═OH
X═O, S, SO, SO₂, Y═OH, Z═H

In an embodiment, the compound(s) of the present technology may be in the form of “B” Compounds:

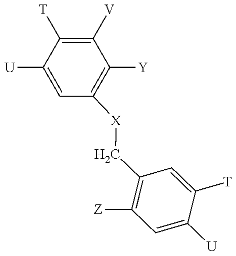

X═O, S, SO, SO₂, Y═Z═OH, T=U═V═H
X═O, S, SO, SO₂, Y═Z═OH, T-U═(CH═CH—CH═CH), V═H
X═O, S, SO, SO₂, Y═Z═OH, T-V═(CH═CH—CH═CH), U═H
X═O, S, SO, SO₂, Y═H, Z═OH, T=U═V═H
X═O, S, SO, SO₂, Y═H, Z═OH, T-U═(CH═CH—CH═CH), V═H
X═O, S, SO, SO₂, Y═H, Z═OH, T-V═(CH═CH—CH═CH), U═H
X═O, S, SO, SO₂, Y═OH, Z═H, T=U═V═H
X═O, S, SO, SO₂, Y═OH, Z═H, T-U═(CH═CH—CH═CH), V═H
X═O, S, SO, SO₂, Y═OH, Z═H, T-V═(CH═CH—CH═CH), U═H

In an embodiment, the compound(s) of the present technology may be in the form of “C” Compounds:

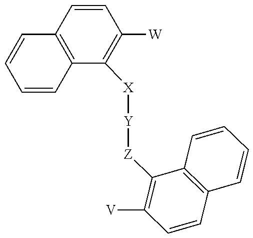

X═S, SO or SO₂, Y═Z═CH2, Y═Z═CH2, W═V=OH
X═S, SO or SO₂, Y═Z═CH2, W═OH, V═H
X═S, SO or SO₂, Y═Z═CH2, W═H, V═OH
X═O, Y═Z═CH2, W=V=OH
X═O, Y═Z═CH2, W═OH, V═H
X═O, Y═Z═CH2, W═H, V═OH
X═Y═Z═CH2, W=V=OH
X═Y═Z═CH2, W═OH, V═H
X═Y═Z═CH2, W═H, V═OH
X═Z=CH2, Y═S, SO or SO₂, W═V=OH
X═Z=CH2, Y═S, SO or SO₂, W═OH, V═H
X═Z=CH2, Y═S, SO or SO₂, W═H, V═OH
X═Z=CH2, Y═O, W═V=OH
X═Z=CH2, Y═O, W═OH, V═H
X═Z=CH2, Y═O, W═H, V═OH

In an embodiment, the compound(s) of the present technology may be in the form of “D” Compounds:

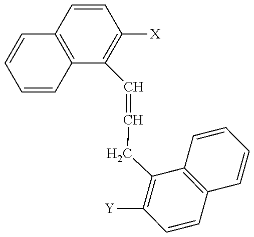

### E- and Z—

X═Y═OH
X═OH, Y═H
X═H, Y═OH

In an embodiment, the compound(s) of the present technology may be in the form of “E” and “F” Compounds respectively:

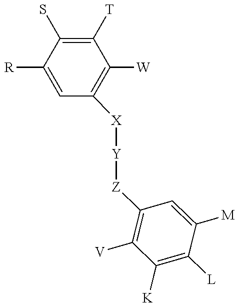

K=L=M=R═S=T=H
K=L=M=R═H, S-T=(CH═CH—CH═CH)
K=L=M=H, R—S═(CH═CH—CH═CH), T=H
K-L=(CH═CH—CH═CH), M=R═S=T=H
L-M=(CH═CH—CH═CH), L=R═S=T=H
K-L=(CH═CH—CH═CH), S-T=(CH═CH—CH═CH), M=R═H
L-M=(CH═CH—CH═CH), S-T=(CH═CH—CH═CH), K=R═H
K-L=(CH═CH—CH═CH), R—S=(CH═CH—CH═CH), M=T=H
L-M=(CH═CH—CH═CH), R—S=(CH═CH—CH═CH), K=T=H
   AND
X═S, SO or SO₂, Y═Z=CH₂, X═S, SO or SO₂, Y═Z=CH₂, W=V=OH
X═S, SO or SO₂, Y═Z=CH₂, W=OH, V═H
X═S, SO or SO₂, Y═Z=CH₂, W=H, V=OH
X═O, Y═Z=CH₂, W=V=OH
X═O, Y═Z=CH₂, W=OH, V═H
X═O, Y═Z=CH₂, W=H, V=OH
X=Y═Z=CH₂, W=V=OH
X=Y═Z=CH₂, W=OH, V═H
X=Y═Z=CH₂, W=H, V=OH
X=Z=CH₂, Y=S, SO or SO₂, W=V=OH
X=Z=CH₂, Y=S, SO or SO₂, W=OH, V═H
X=Z=CH₂, Y=S, SO or SO₂, W=H, V=OH
X=Z=CH₂, Y=O, W=V=OH
X=Z=CH₂, Y=O, W=OH, V═H
X=Z=CH₂, Y=O, W=H, V=OH
   Methods of making the inventive compounds:

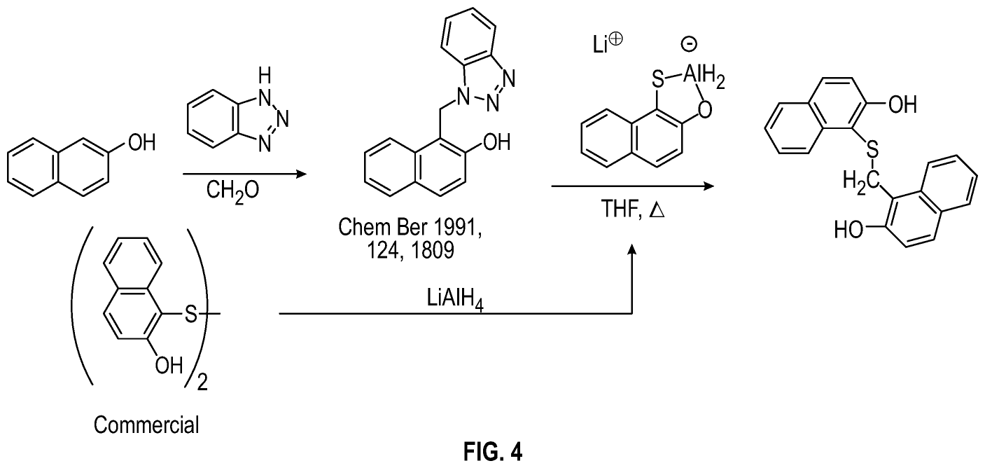

The compounds of the technology can be prepared using chemical synthetic methods such as shown in FIGS. 4-7.

In some embodiments, the compound(s) of the present technology are synthesized based on the protocol shown in FIG. 4. In this example, Compound 1 is synthesized using the precursors, intermediates, and/or reagents identified in FIG. 4 and may be applied to any of the compounds of the present technology. The present technology envisages using this protocol to make similar compounds with the same linkage.

In some embodiments, the following synthesis protocols may be used for synthesizing any one or more of the compounds of the present technology. The present technology envisages using any one of these protocols to make similar compounds with the same linkage.

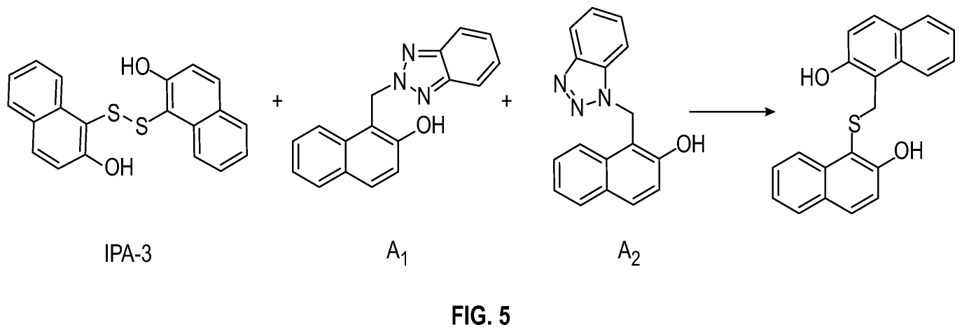

In an embodiment, Compound 1 is synthesized according to the protocol as shown in FIG. 5 as follows:

To a stirred solution of IPA-3 (0.636 g, 1.82 mmol), a mixture of A₁/A₂ (1.0 g, 3.63 mmol) prepared according to the literature method (Katritzky, A. R.: Lan, X.; Lam, J. N, Chem. Ber, 1991, 124, 1809-1817), and KO^{t}Bu (1.02 g, 9.08 mmol) in THF (20 mL) at ambient temperature was added 1M LiAlH₄ in THF (3.6 mL, 3.6 mmol). After agitation at room temperature for 18 h, the reaction was quenched by portion-wise addition of Na₂S₂O₃·5H₂O (5 g), followed by 2N aq. HCl (50 mL), extracted with ethyl acetate and dried on sodium sulfate. Filtration and removal of the solvents under vacuum yielded a crude product that was first purified by chromatography over silica gel (100 g) eluting with a gradient of methyl t-butyl ether in hexane from 0% to 20% yielding 0.84 g of Compound 1 in 70% yield. A second chromatographic purification over silica gel (100 g), eluting with a gradient of methyl t-butyl ether in toluene from 0% to 10%, gave 0.68 g of Compound 1 (0.68 g, 56%).

¹H NMR (300 MHZ, DMSO-d₆), δ: 9.95 (m, 2H), 8.44 (d, J=8.1 Hz, 1H), 8.14 (d. J=8.4 Hz, 1H), 7.90-7.70 (m, 3H), 7.69 (d, J=8.7 Hz, 1H), 7.50-7.40 (m, 2H), 7.34-7.30 (m, 3H), 7.16 (d, J=9.3 Hz, 1H), 4.45 (s, 2H).

¹³C NMR (75 MHZ, DMSO-d₆), δ: 157.2, 153.1, 135.9, 133.1, 130.4, 128.9, 128.7, 128.4, 128.2, 129.0, 126.8, 126.2, 124.6, 123.1, 122.9, 122.5, 118.0, 117.9, 114.7, 112.6, 29.3.

ESI-MS, m/z: 355.2 (M+Na⁺).

ESI-HRMS, m/z: 355.0763: calc for C₂₁H₁₆O₂SNa [M+Na⁺] 355.0769.

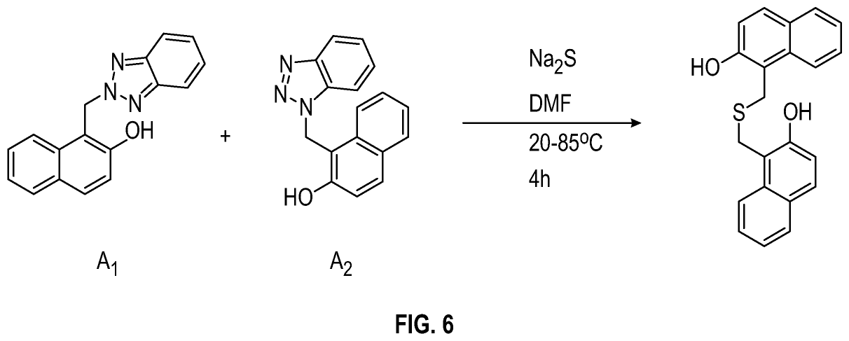

In an embodiment, Compound 3 is synthesized according to the protocol shown in FIG. 6 as follows:

A stirred solution of A₁ and A₂ (2.75 g, 10.0 mmol), prepared according to the literature method (Katritzky, A. R.: Lan, X.: Lam, J. N, Chem. Ber. 1991, 124, 1809-1817), and Na₂S (390.3 mg, 5.0 mmol) in DMF (25 mL) was heated at 85° C. for 4 h. The reaction mixture was cooled to 20° C. and diluted with 90 mL of 2:1 EtOAc/hexanes. The resulting mixture was washed with water (65 mL×2), dried over anhydrous sodium sulfate (6 g), filtered, and concentrated under vacuum to give a residue that was purified by chromatography over silica gel (120 g), eluting with 2-25% EtOAc/hexanes, to give a white solid (1.5 g). Recrystallization from a mixture of EtOAc/hexane (30) mL, 1:2), then gave Compound 3 as a white solid (1.10 g, 64%) with spectral data consistent with the literature values (Vojacek, S.: Beese, K.: Alhalabi, Z.: Swyter, S.: Bodtke, A.: Schulzke, C.: Jung, M.: Sippl, W.: Link, A. Arch. Pharm. 2017, 350, e1700097).

¹H NMR (400 MHZ, DMSO-d₆), δ: 9.80 (s, 2H), 7.77 (d, J=9.0 Hz, 4H), 7.68 (d, J=9.0 Hz, 2H), 7.31 (br t, J=8.1 Hz, 2H), 7.25 (br t, J=8.1 Hz, 2H), 7.17 (d, J=9.1 Hz, 2H), 4.30 (s, 4H). ¹³C NMR (75 MHZ, DMSO-d₆), δ: 152.8, 132.8, 128.4, 129.2, 128.17, 126.0, 123.0, 123.4, 117.9, 115.2, 26.4.

ESI-MS, m/z: 369.1 (M+Na+).

ESI-HRMS, m/z: 369.0920; calc for C₂₂H₁₈O₂SNa [M+Na⁺] 369.0925.

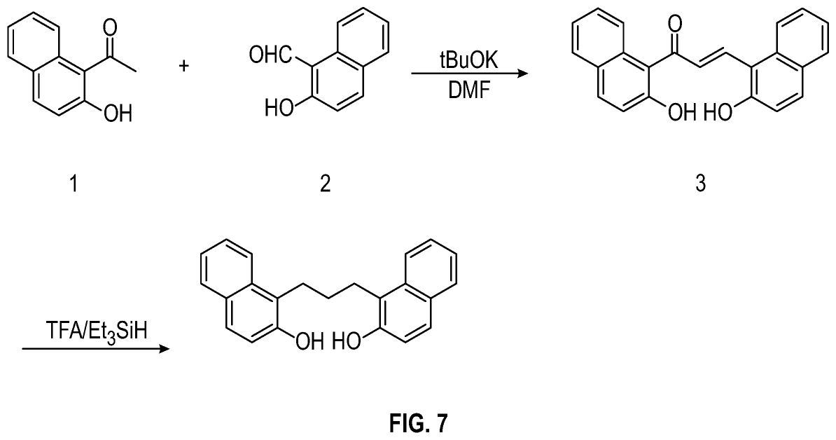

In an embodiment, Compound 4 is synthesized according to the alternative shown in FIG. 7 as follows:

### Step 1: Aldol Condensation

To a stirred solution of IPA-3 (0.636 g, 1.82 mmol), a mixture of A₁/A₂ (1.0 g, 3.63 mmol) A mixture of 1-acetyl-2-naphthol 1 (5.0 g, 0.026 mol) and 2-hydroxy-1-naphthaldehyde 2 (4.6 g, 0.026 mol) were dissolved in anhydrous DMF (55 mL), cooled to 0° C., and treated portion-wise with KO^{t}Bu (11.9 g, 3.95 eq) while maintaining the internal temperature below 5° C. The resulting mixture was stirred below 8° C. for 0.5 h, then heated with stirring to 40° C. for 2 h, and finally to 50° C. for 3.5 h. The reaction was cooled to ambient temperature and was quenched with 3N aqueous HCl (55 mL) to pH 2, and further diluted with 40 ml of water, then extracted with EtOAc (˜250 mL). The EtOAc layer was washed with water (50 mL) and then 20% brine (50 mL) and concentrated to give crude enone 3 (11.7 g).

### Step 2: Enone Reduction to Compound 4

The above crude enone 3 (3.1 g, 9 mmol) was dissolved with stirring in dichloromethane (10 mL) and trifluoroacetic acid (10 mL) to give a dark solution to which was added triethylsilane (8.47 g, 8.0 eq) after which the mixture was heated to 60° C. for 1 h. After cooling to room temperature, the reaction mixture was concentrated under vacuum to ˜⅔ volume, and the resulting slurry was filtered and washed with heptane (˜70 mL). The filtered solid was further washed with water (50 mL) and heptane (10 mL×2) and dried under vacuum to give a crude solid (2.8 g). This solid was stirred in heptane (20 mL) at 55° C. for 2 h, then was filtered off and washed with heptane to give crude Compound 4 (1.75 g) as a solid. Crude Compound 4 (2.3 g) was stirred in MeOH (20 mL) at 35° C. for 0.5 h, then cooled, filtered, and concentrated to give a light brown solid (1.96 g). This solid was suspended in dichloromethane (8.5 mL) and stirred at ambient temperature for 1 h, then was filtered off and washed with 1:1 dichloromethane/heptane (10 mL×2) and heptane (10 mL×2) and dried under vacuum to give Compound 4 (1.29 g, 39%) as an off-white solid.

¹H NMR (400 MHZ, DMSO-d₆), δ: 9.45 (s, 2H), 7.77 (d, J=8.0 Hz, 2H), 7.73 (d, J=8.0 Hz, 2H), 7.59 (d. J=8.0 Hz, 2H), 7.34 (t, J=8.0 Hz, 2H), 7.21 (t, J=8.0 Hz, 2H), 7.14 (d, J=8.0 Hz, 2H), 3.10 (t. J=8.0 Hz, 4H), 1.79 (m, 2H).

¹³C NMR (126 MHz, DMSO-d₆), 152.6, 133.6, 128.9, 128.7, 127.5, 126.4, 123.0, 122.6, 120.1, 118.5, 30.3, 25.2.

ESI-MS, m/z: 328.4 (M+).

ESI-HRMS, m/z: 351.1349; calc for C₂₃H₂₀O₂Na [M+Na⁺] 351.1361.

### Compositions

In an embodiment, the compounds of the present technology may be formulated in any number of compositions containing biocompatible excipients.

Each of the compounds described herein may be present in relatively pure form with adventitious impurities or in impure form. In some embodiments, the purity of the compounds is 90% or greater. In some embodiments, the purity of the compounds is 95% or greater. In some embodiments, the purity of the compounds is 99% or greater. In some embodiments, the purity of the compounds is less than 90%.

The effective dosage amounts of the compound(s) of the present technology may be modulated based on the desired efficacy.

In an embodiment, the compound(s) may represent, for example, greater than or equal to 99.9%, 99%, 95%, 90%, 80%, 70%, 60%, 50%, 40%, 30%, 20%, 10% or 1% by mass or by weight of the total composition or any range between these values.

In an embodiment, each of the compounds of the present technology may also be present in solutions containing the compound and a solvent or in solid compositions containing the compound and other components. An individual compound of the present technology may be present at greater than or equal to 99.9%, 99%, 95%, 90%, 80%, 70%, 60%, 50%, 40%, 30%, 20%, 10% or 1% by mass or at less than 1% by mass of such compositions. Such compositions may also contain two or more of the above described compounds, with the total of these compounds being present at greater than or equal to 99.9%, 99%, 95%, 90%, 80%, 70%, 60%, 50%, 40%, 30%, 20%, 10% or 1% by mass or at less than 1% by mass of such compositions.

In an embodiment, a compound or compounds of the present technology may be present in compositions intended for pharmaceutical use. Such compositions would include other pharmaceutically acceptable components, as well known to one of skill in the art. These compounds may be in liquid or solid form and may be in dosage forms such as liquids for parenteral/intravenous, intramuscular, or enteral/oral administration, including solid compositions in a dosage form such as pills, tablets, and capsules and any other dosage forms as known in the art.

In an embodiment, one or more pharmaceutically acceptable excipients may be used with any of the compositions described in the disclosure.

In an embodiment, the composition may further include IPA-3. Alternatively, the composition may not contain IPA-3.

### Liposomal Formulations

In an embodiment, any one or more of the compounds described herein may be present in a liposomal formulation in which the compound is encapsulated in a lipid-based vehicle such as a liposome.

In an alternative embodiment, any one or more of the compounds described herein may be present in a composition that does not comprise a liposomal formulation.

In some embodiments, the liposomes are formed of one or more of 1,2-distearoyl-sn-glycero-3 phosphatidylcholine (DSPC), 1,2-distearoyl-sn-glycero-phosphatidylethanolamine (DSPE), and 1,2-distearoyl-sn glycero-3-phosphoethanolamin-N-[poly(ethyleneglycol) 2000 (DSPE-PEG) and can include a sterol, for example, cholesterol.

Other lipids and components useful in preparing the disclosed nanoparticulate compositions are known in the art. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines: phosphatidylserine (PS), phosphatidylglycerol, phosphatidylinositol (PI): glycolipids: sphingophospholipids such as sphingomyelin and sphingoglycolipids (also known as 1-ceramidylglucosides) such as ceramide galactopyranoside, gangliosides and cerebrosides: fatty acids, sterols, containing a carboxylic acid group for example, cholesterol: 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC).

Suitable cationic lipids in the liposomes include, but are not limited to, N-[1-(2,3-dioleoy loxy) propyl]-N, N, N-trimethyl ammonium salts, also referred to as TAP lipids, for example methylsulfate salt. Suitable TAP lipids include, but are not limited to, DOTAP (dioleoyl), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes. N-| 1-(2,3-diolovloxy) propyl]-N, N-dimethy amine (DODAP), 1,2-diacyloxy-3-dimethylammonium propanes. N-[1-(2,3-dioley loxy) propyl]-N, N, N-trimethylammonium chloride (DOTMA), 1,2-dialkyloxy-3-dimethylammoniumpropanes, dioctadecylamidoglycylspermine (DOGS),3-[N—(N′, N′-dimethylamino-ethane carbamoyl| cholesterol (DC-Chol): 2,3-dioleoyloxy-N-(2-(sperminecarboxamido) ethyl)-N, N-dimethyl-1-propanaminium trifluoro-acetate (DOSPA), β-alanyl cholesterol, cetyl trimethyl ammonium bromide (CTAB), diC14-amidine. N-tert-butyl-N′-tetradecyl-3-tetradecylamino-propionamidine. N-alpha-trimethylammonioacetyl) didodecyl-D-glutamate chloride (TMAG), ditetradecanoyl-N-trimethylammonio-acetyl) diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1.4 butanediammonium iodide.

In one embodiment, the cationic lipids can be 1-[2-(acyloxy) ethyl]2-alkyl (alkenyl) -3-(2-hydroxyethyl)-imidazolinium chloride derivatives, for example, 1-[2-(9) (Z)-octadecenoyloxy) ethyl)-2-(8 (Z)-hepta (DOTIM), and 1-[2-(hexadecanoyloxy)ethyl]-2-pentadecyl 3-(2-hydroxyethyl) imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-di methyl-hydroxypropyl ammonium bromide (DORIE-HP), 1,2-dioleyl-oxy-propyl-3-dimethyl-hydroxybutylnium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids can be formed from a combination of more than one lipid. For example, a charged lipid may be combined with a lipid that is non-ionic or uncharged at physiological pH. Non-ionic lipids include cholesterol and DOPE (1,2-dioleolylglycerylphosphatidylethanolamine) and other non-ionic lipids known in the art.

A departure from the embodiment is the inclusion of specific lipids known to induce liposomal entry into cancer cells. This will be achieved by including additional lipids whose presence and levels align with those identified in cancer lipids profiles as determined by performing lipidomic analysis of multiple types of cancer cells. These include stoichiometric ratios of 36:1 phosphatidylcholine (PC), 18:0 to 22:6 PC, 34:1 sphingomyelin (SM), oxidized phosphatidylethanolamine (Ox-PE), and 20:4 lysophosphatidylcholine (20:4 LPC).

The stoichiometric percentage of the individual lipids mentioned above within the liposomal formulation can be altered from 1% to 80% of the total lipid amount. For example, the stroichiometric percentage of any component lipid can be 1%, 2%, 5%, 10%, 15%, 20%, 25%, 30%, 40%, 50%, 60%, 70%, 80%, or any range between these values.

In some embodiments, the nanoparticulate composition includes a sterically stabilized liposome including cholesterol, and phospholipids such as DSPC and DSPE-PEG. For example, lipid-based delivery vehicles such as liposomes can include DSPC, DSPE-PEG, and cholesterol in a stoichiometric ratio of 9:1:5. In some embodiments, the PEG is PEG-2000.

In one method of preparing the liposomes, formulations will be created by dissolving cholesterol, phospholipids, DSPE-PEG in chloroform, and the active compound of the technology in ethanol, mixing these together, and evaporating the solvents under a vacuum. The formed thin film can be hydrated and suspended in ammonium phosphate buffer or phosphate saline buffer (PBS) to achieve a final lipid concentration of about 10 μmol/ml. The formulation can be subjected to liquid nitrogen freeze-thaw cycles above the phase transition temperature of the primary lipid and passed through a Lipex extruder using double-stacked polycarbonate membranes. Excess unencapsulated drugs and lipids can be eliminated using dialysis, by methods known in the art.

Each of the above-described compounds of the technology may be present as a component of a chemical library, which is a collection of chemical compounds intended for use in high throughput screening. Physical forms of such libraries are well known in the art. For example, one of the above-described compounds could be present in one well of a microtiter plate, in which the other wells contain different chemical compounds.

### Methods of Use

The above-described compounds and compositions can be useful when administered to patients suffering from certain diseases or disorders.

In an embodiment, effective amounts of the compounds of the present technology are directed at treating diseases or disorders associated with p21-activated kinase-related diseases or disorders. In a preferred embodiment, the diseases or disorders are associated with PAK1.

In an embodiment, the present technology is directed to a method of treating a disease or disorder associated with PAK1. The method includes administering an effective amount of the compounds and/or compositions of the present technology. “Effective amount” means any amount of a compound and/or composition in a single dosage form that is capable of inhibiting PAK1 for treating the associated disease or disorder.

In some embodiments, the PAK1-related diseases or disorders include, but are not limited to: different types of hematological (e.g., multiple myeloma, Leukemia, and ibrutinib (Imbruvica)-resistant and parental chronic lymphocytic leukemia, etc.): solid tumors (e.g. prostate, breast, pancreatic, head & neck, NSCLC, melanomas/skin cancer, and liver cancers, etc.): cancer metastasis: myelodysplastic syndrome: cardiac hypertrophy: organ fibrosis (e.g. heart, lung, kidney, and liver, etc.): diseases relating to vascular oversufficiency (e.g., anti-angiogenic therapy, diabetic retinopathy, etc.): infectious/inflammatory disorders (e.g., viral and bacterial infections, allergen-induced disorders); and neurological and mental disorders (e.g., Alzheimer disease. Fragile X syndrome, and Huntington's disease, etc.).

In a preferred embodiment, the method treats cancerous cells by administering a compound or composition of the presenting technology.

Administration of the compositions of the technology can be performed using methods known in the art. For example, parenteral/intravenous and enteral/oral administration can be performed among others as long as the dosage form is biocompatible.

Any of the compounds or compositions as described herein may be used with the methods of use as also described herein.

The term “substantially” as used herein, unless otherwise indicated, means to a great extent, for example, greater than about 95%, greater than about 99%, greater than about 99.9%, greater than 99.99%, or even 100% of a referenced characteristic, quantity, etc. as pertains to the particular context (e.g., substantially pure, substantially the same, and the like).

The following examples illustrate various examples of continuous mixing systems for forming an emulsion in accordance with the present technology. In addition, the following examples illustrate various methods for controlling resulting bead properties from an emulsion made with a continuous mixing system in accordance with the present technology.

All methods described herein can be performed in any suitable order unless otherwise indicated herein or otherwise clearly contradicted by context. The use of any and all examples, or exemplary language (e.g., “‘such as’”) provided herein, is intended merely to better illustrate the materials and methods and does not pose a limitation on the scope unless otherwise claimed. No language in the specification should be construed as indicating any non-claimed element as essential to the practice of the disclosed materials and methods.

It will be readily apparent to one of ordinary skill in the relevant arts that suitable modifications and adaptations to the compositions, methods, and applications described herein can be made without departing from the scope of any examples or aspects thereof. The compositions and methods provided are exemplary and are not intended to limit the scope of the claimed embodiments. All of the various embodiments, aspects, and options disclosed herein can be combined in all variations. The scope of the compositions, formulations, methods, and processes described herein include all actual or potential combinations of embodiments, aspects, options, examples, and preferences herein.

Although the technology herein has been described with reference to particular embodiments, it is to be understood that these embodiments are merely illustrative of the principles and applications of the present technology. It will be apparent to those skilled in the art that various modifications and variations can be made to the method and apparatus of the present technology without departing from the spirit and scope of the technology. Thus, it is intended that the present technology include modifications and variations that are within the scope of the appended claims and their equivalents.

Reference throughout this specification to “one embodiment.” “certain embodiments,” “one or more embodiments” or “an embodiment” means that a particular feature, structure, material, or characteristic described in connection with the embodiment is included in at least one embodiment of the technology. Thus, the appearance of phrases such as “‘in one or more embodiments.’” “‘in certain embodiments,’” “‘in one embodiment’” or “‘in an embodiment’” in various places throughout this specification are not necessarily referring to the same embodiment of the technology. Furthermore, the particular features, structures, materials, or characteristics may be combined in any suitable manner in one or more embodiments. Any ranges cited herein are inclusive.

Aspects of the present technology are more fully illustrated with reference to the following examples. Before describing several exemplary embodiments of the technology, it is to be understood that the technology is not limited to the details of construction or process steps set forth in the following description. The technology is capable of other embodiments and of being practiced or being carried out in various ways. The following examples are set forth to illustrate certain aspects of the present technology and are not to be construed as limiting thereof.

### EXAMPLES

Compound 1, Compound 3, and Compound 4 were tested for their anti-cancer efficacy on human metastatic prostate cancer (PC3) cells. Because of their structural similarity with IPA-3, a PAK1 inhibitor demonstrated to inhibit prostate cancer cells function in vitro, the anti-cancer efficacy of the novel compounds was compared with IPA-3 as a positive control and a reference comparison. Vehicle solvent (DMSO) was used as a negative control.

### Example 1: MTT Assay

A MTT (3-[4,5-dimethylthiazol-2-yl]-2,5 diphenyl tetrazolium bromide) assay was used to assess cancer cell metabolic activity and proliferation.

The protocol was as follows:
A total of 4000 PC3 cells were seeded per well in 96-well plates. Following attachment, cells were treated with vehicle control DMSO, IPA3, Compound 1, Compound 3, and Compound 4 at various doses as indicated for 24 h. Growth inhibition was determined by MTT [3-(4,5-dimethylthiazol-2-yl)-2,5-diphenyltetrazolium bromide] (Sigma-Aldrich, St. Louis, MO, USA; catalog No. M2128) assay. The MTT solution was added to the media at a final concentration of 0.8 mg/mL and cells were incubated at 37° C. for 2 hours. After aspiration of media, formazan crystals were dissolved in 100 μl of DMSO. Optical densities were measured at 570 nm using a SynergyHT plate reader (BioTek. Winooski. WI. USA). To calculate IC50 values. GraphPad Prism Software was used (GraphPad Software. San Diego, CA. USA).

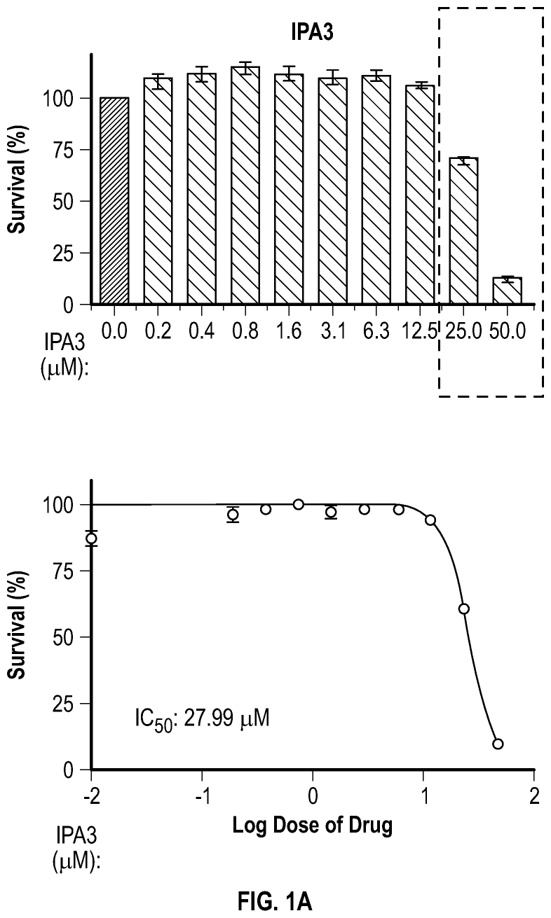

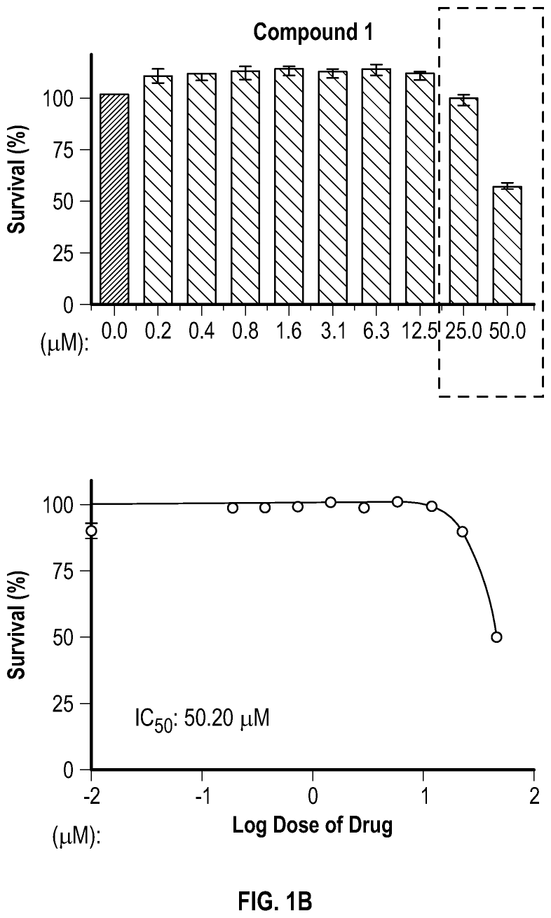

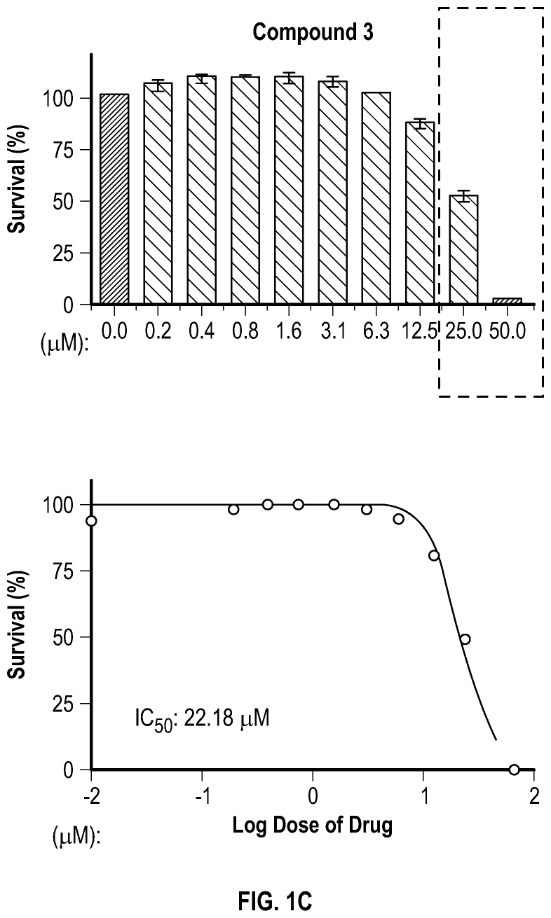

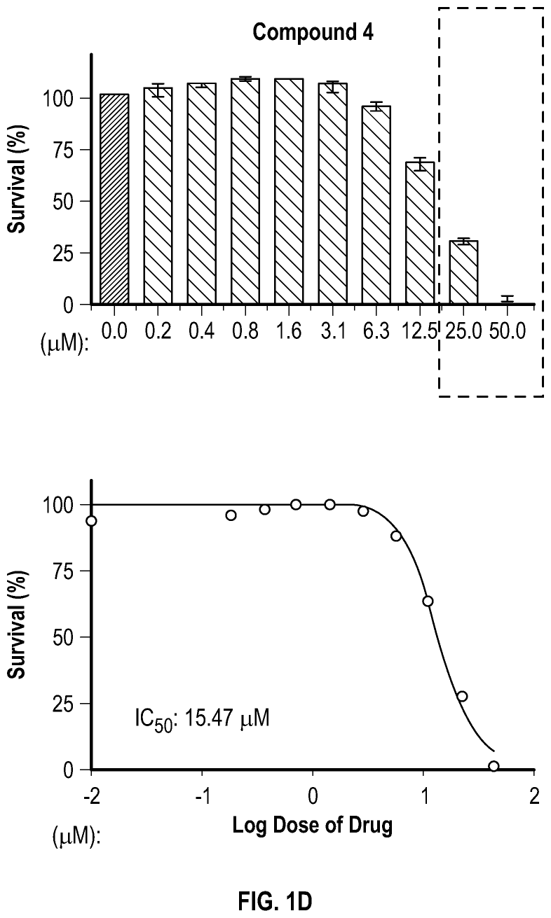

As shown in FIGS. 1A-1D, the results demonstrated that IPA3 and all three compounds (Compound 1. Compound 3, and Compound 4) have anti-cancer effects in reducing the metabolic activity and hence the cell proliferation of PC3 cells. Whereas the IC50 value of Compound 1 was surprisingly higher compared to the IPA3 treated arm (50.20 μM versus 27.99 μM, respectively). Compound 3 and Compound 4 demonstrated superior efficacy and lower IC50 compared to IPA3 (22.18 μM and 15.47 μM, respectively). The study demonstrated the efficacy of Compound 1. Compound 3, and Compound 4 in suppressing prostate cancer cell proliferation and that the effect of Compound 3 and Compound 4 is superior to PAK1 inhibitor. IPA3.

These results are especially surprising given that the disulfide bridge in IPA-3 was well regarded in the art as being necessary for anti-cancer efficacy. For example, in Deacon. Sean W . . . et al. Chemistry & biology 15.4 (2008): 322-331, the authors tested a number of structurally related compounds yet none inhibited PAK1 kinase activity as potently as IPA-3. See Deacon at page 324, col 1, second full paragraph. Moreover, the authors concluded that the specific mechanism of PAK1 inhibition by IPA-3 was based on the role of the disulfide bond in IPA-3. In contrast, the compounds of the present technology (e.g., Compound 1. Compound 3, and Compound 4) are more structurally stable by lacking a disulfide bond and were even more effective than IPA-3 despite the literature describing the disulfide bond as a critical component for PAK1 inhibition.

### Example 2: Cell Motility Assay

The motility of cancer cells is an index of their ability to come out of the primary tumor, invade, and metastasize to distant tissues. MetasTx tested Compound 1. Compound 3, and Compound 4 for their inhibitory effect on human metastatic prostate cancer (PC3) cells by using a ‘cell-monolayer scratch assay”, a reliable technique to determine the rate of cancer cell motility.

The protocol was as follows:

A total of 0.5×105 PC3 cells were plated per well on 6 well plates to obtain 95% confluent monolayers of cells after overnight incubation. The following day, a linear scratch (wound) was made using a 200 μl pipette tip and the detached cells were washed away by sterile phosphate-buffered saline (PBS). Images of scratches were captured from three different locations under a phase-contrast microscope at 4× magnification. The monolayers were treated with the desired doses of vehicle or drugs (half of the IC50 doses determined from the MTT assay). The cells were allowed to migrate for 36 hours, and images of the wound were re-captured from the same locations. The percentage of healed areas was determined using the NIH ImageJ software by measuring the distance of imported images in Microsoft Office PowerPoint (Microsoft Office Inc.) at the same magnification.

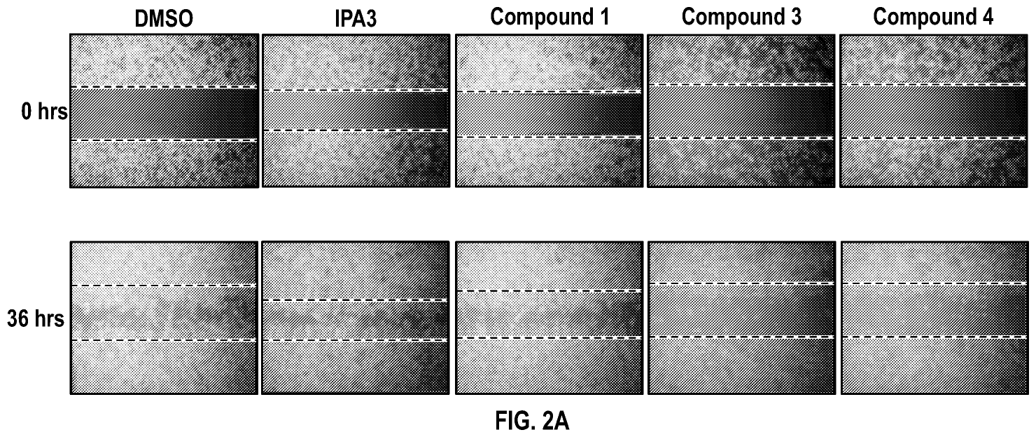

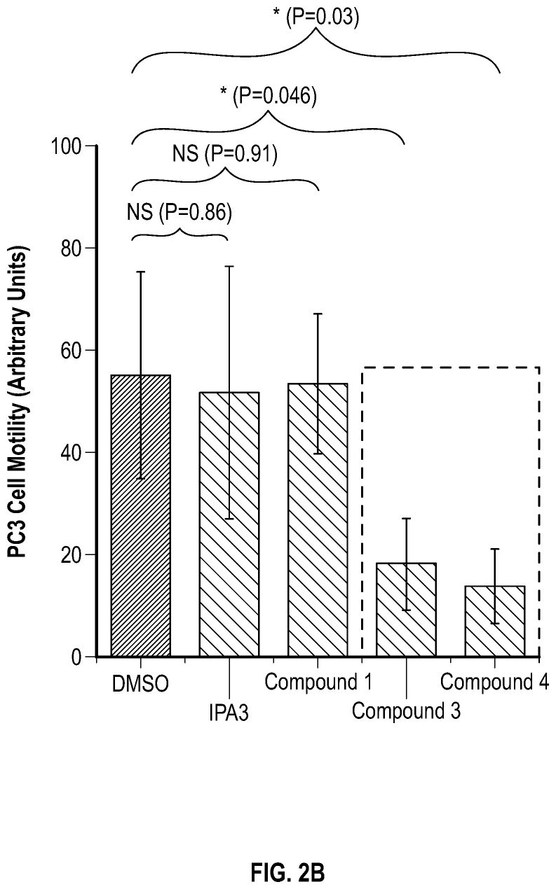

As shown in FIGS. 2A and 2B, the analysis of PC3 cell motility demonstrated significant anti-cancer cell motility effects of Compound 3 and Compound 4 compared to vehicle (DMSO)-treated control. Treatment with IPA3 or Compound 1 did not show significant inhibition of PC3 cell motility, suggesting the superior efficacy of Compound 3 and Compound 4 on prostate cancer cell motility.

### Example 3: Apoptosis and Necrosis Assay

The anti-cancer efficacy of a drug was also evaluated based on the ability of the compound to induce apoptosis and/necrosis in cancer cells. Hence. Compound 1. Compound 3, and Compound 4 were tested based on their ability to induce apoptosis and necrosis in vitro. The protocol was as follows:

The apoptotic death of cells was determined using an Annexin V-PE apoptosis detection kit (Cat #559763; BD Biosciences. CA. USA) according to the manufacturer's protocol. In brief. PC3 cells were treated with DMSO (0.3%). IPA3. Compound 1. Compound 3 and Compound 4 for 24 hours. At the end of treatment, cells were trypsinized, washed with PBS and stained with annexin V-Phycoerythrin (PE) and 7-Amino-Actinomycin (7-AAD). The stained cells were analyzed using a flow cytometer within 1 h of staining.

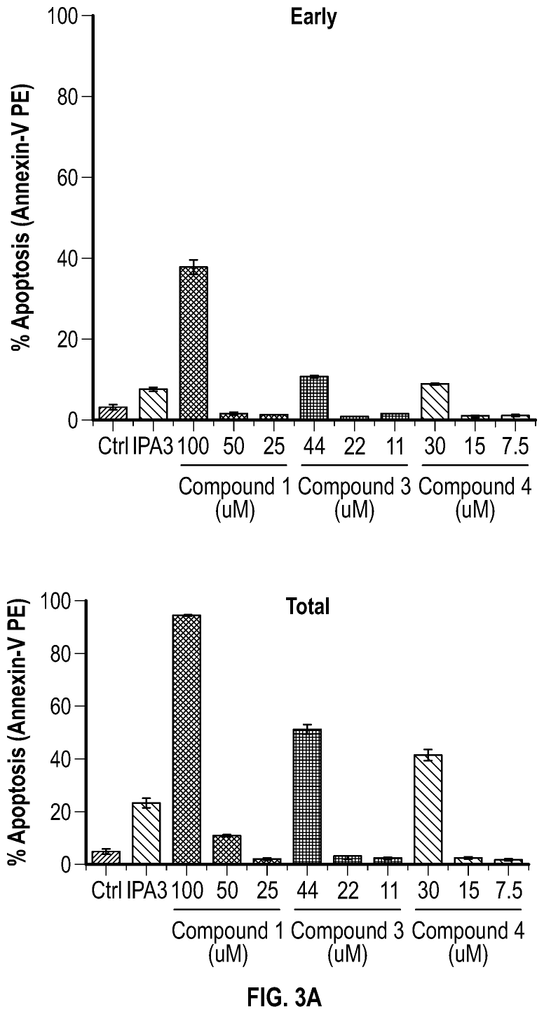

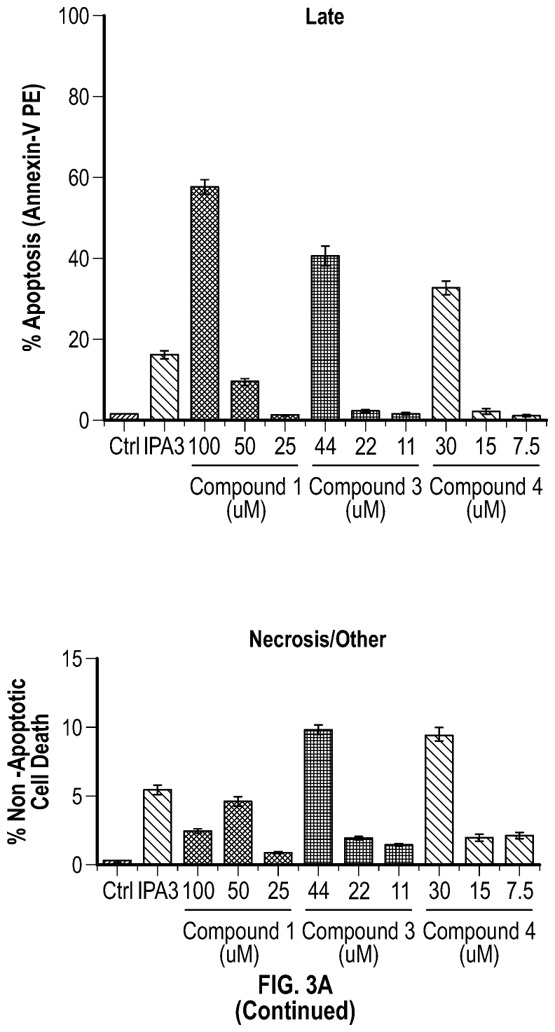

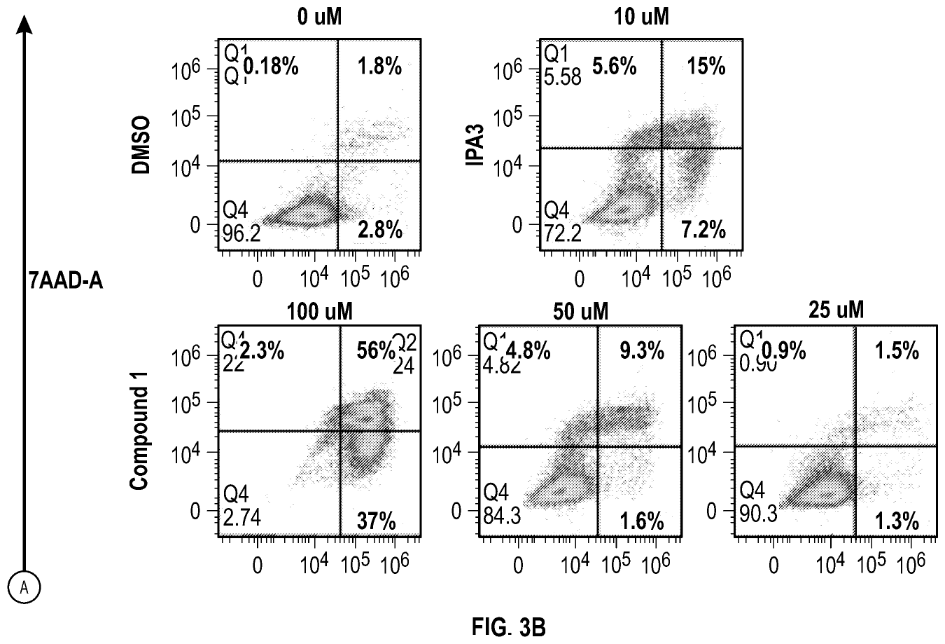

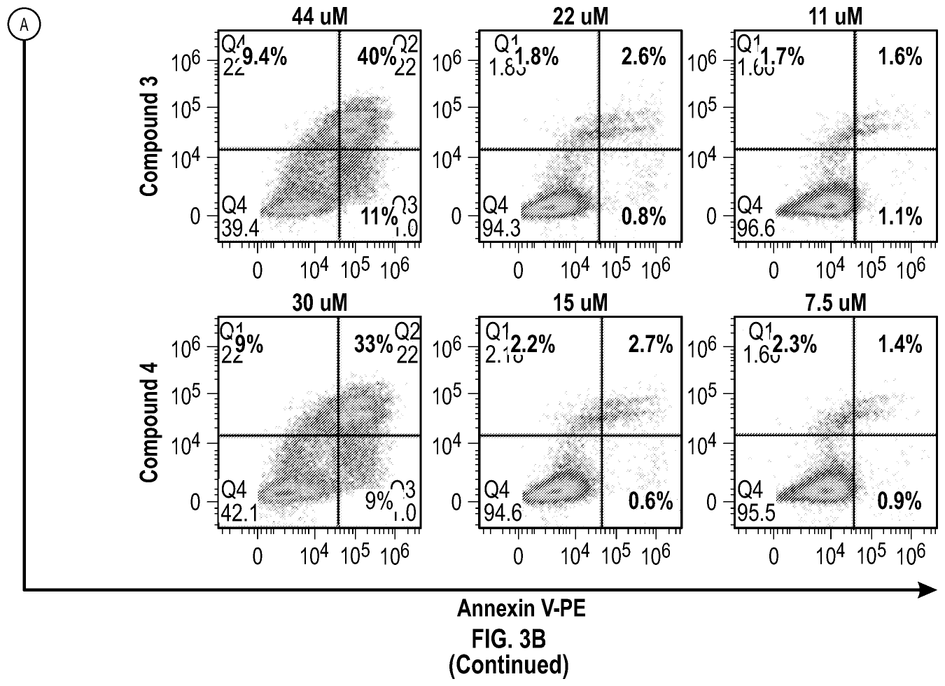

As shown in FIGS. 3A and 3B, the analysis of PC3 cell apoptosis and necrosis revealed a robust induction of apoptosis and necrosis (cell death independent of the apoptotic pathways) by IPA-3. Compound 1. Compound 3 and Compound 4 compared to vehicle (DMSO)-treated control. As evident from the other functional assays, treatment with Compound 3 and Compound 4 had superior efficacy in inducing total cell death compared to IPA-3.

Aspects of the present technology are more fully illustrated with reference to the following examples. Before describing several exemplary embodiments of the technology, it is to be understood that the technology is not limited to the details of construction or process steps set forth in the following description. The technology is capable of other embodiments and of being practiced or being carried out in various ways. The following examples are set forth to illustrate certain aspects of the present technology and are not to be construed as limiting thereof.

Further modifications and alternative aspects of the disclosure will be apparent to those skilled in the art in view of this description. Accordingly, this description is to be construed as illustrative only and is for the purpose of teaching those skilled in the art the general manner of carrying out the present technology. It is to be understood that the forms of the technology shown and described herein are to be taken as examples. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the following claims.

When used in this specification and claims, the terms “comprises” and “comprising” and variations thereof mean that the specified features, steps or integers are included. The terms are not to be interpreted to exclude the presence of other features, steps or components. The technology comprises, consists of, or consists essentially of the disclosed and claimed features.

The technology may also broadly consist or comprise of the parts, elements, steps, examples and/or features referred to or indicated in the specification individually or collectively in any and all combinations of two or more said parts, elements, steps, examples and/or features. In particular, one or more features in any of the embodiments, examples and aspects described herein may be combined with one or more features from any other embodiments, examples and aspects described herein. Although certain example embodiments of the disclosure have been described, the scope of the appended claims is not intended to be limited solely to these embodiments. The claims are to be construed literally, purposively, and/or to encompass equivalents.

What is claimed is:

1. A compound having the structure: 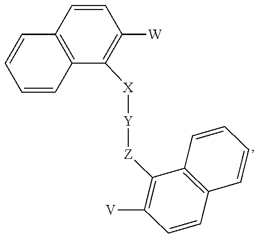
or a pharmaceutically acceptable salt thereof,
wherein W and V are each independently H or OH;
wherein X and Y are each independently an attachment site, methine, methylene, N, N-alkyl, N-acyl, N-sulfonyl, O, S, CO, or SO₂, with the provisos that X and Y cannot both be an attachment site, O, S, or SO₂ and that if X or Y is methine, X and Y are both methines to form a double bond;
wherein the alkyl group of the N-alkyl and N-acyl groups is a C1-C6 alkyl group that is linear or branched, unsubstituted or substituted, with one or more substitutions selected from the group consisting of hydroxyl groups, halogens, cyano groups, amines, and mixtures thereof; and
wherein Z is CHR where R is H or C1-C6 alkyl, wherein the C1-C6 alkyl group of Z is linear or branched, unsubstituted or substituted, with one or more substitutions selected from the group consisting of hydroxyl groups, halogens, cyano groups, amines, and mixtures thereof.

2. The compound of claim 1, wherein X is an attachment site.

3. The compound of claim 1, wherein the compound is Compound 1: 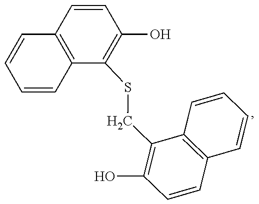 [SMILES: Oc1ccc2ccccc2c1CSc1c(O)ccc2ccccc12]
or a pharmaceutically acceptable salt thereof.

4. The compound of claim 1, wherein the compound is Compound 3: 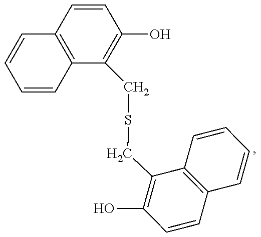 [SMILES: Oc1ccc2ccccc2c1CSCc1c(O)ccc2ccccc12]
or a pharmaceutically acceptable salt thereof.

5. The compound of claim 1, wherein the compound is Compound 4: 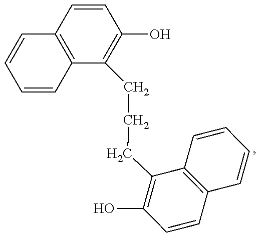 [SMILES: Oc1ccc2ccccc2c1CCCc1c(O)ccc2ccccc12]
or a pharmaceutically acceptable salt thereof.

6. A composition comprising:
the compound of claim 1 or a pharmaceutically acceptable salt thereof; and
one or more pharmaceutically acceptable excipients.

7. The composition of claim 5, wherein the composition comprises an effective amount of the compound of claim 1.

8. The composition of claim 5, wherein the composition comprises a liposomal formulation.

9. The composition of claim 5, wherein the composition does not comprise a liposomal formulation.

10. A method of treating a disease or disorder associated with PAK1 comprising administering an effective amount of the compound of claim 1.

11. The method of claim 7, wherein the compound is Compound 1: 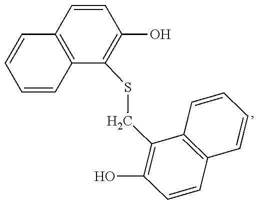 [SMILES: Oc1ccc2ccccc2c1CSc1c(O)ccc2ccccc12]
or a pharmaceutically acceptable salt thereof.

12. The method of claim 7, wherein the compound is Compound 3: 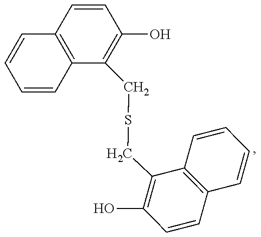 [SMILES: Oc1ccc2ccccc2c1CSCc1c(O)ccc2ccccc12]
or a pharmaceutically acceptable salt thereof.

13. The method of claim 7, wherein the compound is Compound 4: 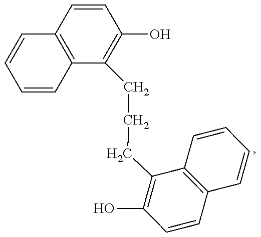 [SMILES: Oc1ccc2ccccc2c1CCCc1c(O)ccc2ccccc12]
or a pharmaceutically acceptable salt thereof.

14. A method of treating cancerous cells comprising administering an effective amount of the compound of claim 1.
